(19) United States
(12) Patent Application Publication
(10) Pub. No.: US 2017/0181792 A1
(43) Pub. Date: Jun. 29, 2017

(54) LEFT-RIGHT PARALLEL LOOP BIPOLAR ELECTRODE

(71) Applicant: SCANMED (CHINA) LTD., Zhuhai, Guangdong (CN)

(72) Inventor: Min Lin, Zhuhai, Guangdong (CN)

(21) Appl. No.: 15/308,613

(22) PCT Filed: May 14, 2016
(86) PCT No.: PCT/CN2016/082156

§ 371 Date: Nov. 3, 2016

(30) Foreign Application Priority Data
Jun. 24, 2015 (CN) 201510355477.6

(51) Int. Cl. A61B 18/14

(52) U.S. Cl. CPC A61B 18/1485; A61B 18/14; A61B 2018/1407

(57) ABSTRACT

The invention provides a left-right parallel loop bipolar electrode. At its front-section part, a metal sleeve and a rigid tube are arranged in parallel at an interval on left and right, two insulating tubes are fixed at heads of the metal sleeve and the rigid tube respectively, one end of a positive electrode is connected with a wire, led from the rigid tube, with an insulating layer in the corresponding insulating tube, and another wire with an insulating layer is welded with the metal sleeve. At its rear-section part, a tail end of the metal straight tube is connected with a binding post, the binding post is connected with a double-core wire, and the two wires with the insulating layers penetrate through the metal straight tube to be correspondingly connected with the double-core wire respectively; the other end of the positive electrode is fixed in the other insulating tube.

### TECHNICAL FIELD OF THE INVENTION

The invention relates to the technical field of medical apparatuses and instruments, and in particular to an electrode for a resectoscope for a minimally invasive operation.

### BACKGROUND OF THE INVENTION

Prostatic hypertrophy and hyperplasia are common diseases of medium-elderly men at extremely high incidence, and excision is the most effective treatment method. A conventional open operation is to make a small incision in the lower abdomen of a patient and then incise the urinary bladder to take the prostate out, which seriously injures a normal physiological structure of the urethra. Moreover, most of patients suffering from prostatic hyperplasia are the elderly, they usually suffer from other serious diseases at the same time and it is hard for them to bear traumas brought by open operations, so that various minimally invasive transurethral prostatic hyperplasia treatment methods attract great attentions of urologists. Transurethral prostatic resection with resectoscopes is developed by European and American developed counties, and such an operation does not require incision in a human body so as to greatly reduce traumas, and is known as a “gold standard” for prostatic hyperplasia treatment at present.

A coaxial loop bipolar electrode, disclosed in inventive patent CN201492495U, is commonly used at present. Referring to FIG. 1, a metal tube of a front section of the parallel loop bipolar electrode is forked, a ceramic tube is assembled at a port of the metal tube, and a rear end consists of two parallel steel tubes 31; a positive electrode 35 penetrates through the steel tubes 31 to be connected with a wire 36, and in the electrode, the parallel steel tubes form a loop electrode; and an opening of a corresponding waterproof plug 34 is unlikely to be consistent with the parallel steel tubes in shape. The opening of the waterproof plug is not round but irregularly shaped and the waterproof plug may not be ensured to completely contact with the steel tubes without any slit in a moving process, which may cause the problem of water leakage; and frictional force between the steel tubes sleeved with insulating tubes and the waterproof plug is increased, so that resistance to forward and backward movement of the electrode is increased to cause influence on operation of a doctor and operation fatigue. In addition, the parallel steel tubes form the loop electrode, and although the insulating tubes are arranged on their surfaces, the insulating tubes may be abraded to reduce voltage resistance to bring electric leakage risks after a long operation time. Moreover, the parallel steel tubes occupy a space larger than that occupied by a single tube, so that saline water in an inner sheath of a resectoscope is blocked to flow, and a convection condition of the saline water is not ideal.

A front-section part of another bipolar electrode disclosed in patent EP1072230A1 is consistent with the shape described in CN201492495U, while a rear-section part is different, the parallel steel tubes are replaced with a round steel tube, and the opening of the waterproof plug is correspondingly round, so that the phenomenon of water leakage of the resectoscope may be effectively improved; and however, the round steel tube is still used as a loop electrode, so that electric leakage risks caused by abrasion and voltage resistance reduction of an insulating tube on a surface of the steel tube may not be eliminated. Moreover, after the insulating tube is added, resistance to the electrode is increased, and a size of the electrode may also be enlarged.

In addition, incision accuracy and bleeding control effects of a front-rear double-ring bipolar loop electrode on the market are both poorer than those of a single-ring electrode; and although an upper-lower double-ring bipolar loop electrode solves the abovementioned problems, but it may influence an operation field and further influence operation of a doctor.

### SUMMARY OF THE INVENTION

In view of this, a purpose of the invention is to provide a novel parallel loop bipolar electrode capable of solving both problems of water leakage and electric leakage.

The left-right parallel loop bipolar electrode of the invention reduces a contact area and parts of a loop electrode and a human body as much as possible, reduces stimulation effects of current on the human body, also maintains advantages of a single-ring incision operation, overcomes the shortcomings of poor sealing, non-ideal convection, high electrode resistance and the like of a bipolar electrode which is used more on the market, and solves the problem of electric leakage caused by abrasion to an insulating layer.

The purpose of the invention is achieved by the following technical solutions.

A left-right parallel loop bipolar electrode includes:

a positive electrode, two insulating tubes, a metal sleeve serving as a negative electrode, a rigid tube, two insulating sleeves which are forked by separation at one end and folding at the other end, a metal straight tube with an enlarged opening, two wires with insulating layers, a double-core wire and a binding post;

at a front-section part of the bipolar electrode, the metal sleeve and the rigid tube are arranged in parallel at an interval on left and right, the two insulating tubes are fixed at heads of the metal sleeve and the rigid tube respectively, one end of the positive electrode is connected with the wire, led from the rigid tube, with the insulating layer in the corresponding insulating tube, and the other wire with the insulating layer is welded with the metal sleeve; rear sections of the metal sleeve and the rigid tube are arranged in the two insulating sleeves in a sleeving manner respectively; at a rear-section part of the bipolar electrode, rear sections, which are folded, of the two insulating sleeves are sleeved and covered at the enlarged opening of the metal straight tube; a tail end of the metal straight tube is connected with the binding post, the binding post is connected with the double-core wire, and the two wires with the insulating layers penetrate through the metal straight tube to be correspondingly connected with the double-core wire respectively; the other end of the positive electrode is fixed in the other insulating tube; and

high-frequency energy forms a loop in a working medium through the two wires with the insulating layers in the metal straight tube.

Another left-right parallel loop bipolar electrode includes:

a positive electrode, two insulating tubes, a metal sleeve serving as a negative electrode, an insulating rigid tube, an insulating sleeve, a metal straight tube with an enlarged opening, two wires with insulating layers, a double-core wire and a binding post;

at a front-section part of the bipolar electrode, the metal sleeve and the rigid tube are arranged in parallel at an interval on left and right, the two insulating tubes are fixed at heads of the metal sleeve and the insulating rigid tube respectively, one end of the positive electrode is connected with the wire, led from the rigid tube, with the insulating layer in the corresponding insulating tube, and the other wire with the insulating layer is welded with the metal sleeve; a rear section of the metal sleeve is arranged in the insulating sleeve in a sleeving manner; at a rear-section part of the bipolar electrode, a rear section of the insulating rigid tube is folded with a rear section of the insulating sleeve, and its periphery is sleeved and covered at the enlarged opening of the metal straight tube; a tail end of the metal straight tube is connected with the binding post, the binding post is connected with the double-core wire, and the two wires with the insulating layers penetrate through the metal straight tube to be correspondingly connected with the double-core wire respectively; the other end of the positive electrode is fixed in the other insulating tube; and

high-frequency energy forms a loop in a working medium through the two wires with the insulating layers in the metal straight tube.

Preferably, the left-right parallel loop bipolar electrode further includes an electrode fixing clip, which is welded on an outer surface of the metal straight tube and used for matching with a rod of an endoscope.

Preferably, an external diameter of the metal sleeve is 1 mm-1.7 mm, and an external diameter of the metal straight tube is 1.5-2.2 mm.

Preferably, an exposed front-section part, uncovered by the insulating sleeve, of the metal sleeve is a loop electrode, and a surface area of the loop electrode is 5 to 16 times an electrode surface area of the cambered positive electrode.

Preferably, a shape of the positive electrode is a semicircle, a cylinder or a spade.

Preferably, the insulating tubes are ceramic insulating tubes.

Compared with a conventional art, the invention has the following advantages:

the high-frequency energy of the left-right parallel loop bipolar electrode of the invention forms the loop through the two wires with the insulating layers in the metal straight tube, and the metal straight tube does not serve as a loop electrode and is not electric, so that no insulating tube is required to be arranged on its periphery in the sleeving manner, and there are no electric leakage risks caused by abrasion and voltage resistance reduction of an insulating sleeve on a surface of a round steel tube due to the fact that the round steel tube in a bipolar electrode disclosed in EP1072230A1 in the background serves as a loop electrode; in addition, the insulating layers and the metal straight tube form two protection layers on the peripheries of the wires, so that electric leakage risks caused by abrasion of the protection layers are greatly reduced; moreover, a waterproof plug of the invention is closely arranged on a periphery of the metal straight tube in the sleeving manner, and the metal straight tube of the invention (other positions except the enlarged opening) is a round tube, and is regularly shaped, so that there is no gap between the waterproof plug and the metal straight tube, and the phenomenon of water leakage caused by the fact that an opening of a waterproof plug of a parallel loop bipolar electrode disclosed in inventive patent CN201492495U in the background is irregularly shaped and may not be sealed with a steel tube may be avoided. Therefore, the invention is safer in use and suitable for large-scale popularization.

### BRIEF DESCRIPTION OF THE DRAWINGS

FIG. 1 is an overall structure diagram of a bipolar electrode of a resectoscope in the conventional art according to the background of the invention;

FIG. 2 is an overall structure diagram of a parallel loop bipolar electrode according to an embodiment of the invention;

FIG. 3 is a top overall view of a front-section part of a parallel loop bipolar electrode according to an embodiment of the invention;

FIG. 4 is a side overall view of a front-section part of a parallel loop bipolar electrode according to an embodiment of the invention; and

FIG. 5 is a section view of a front-section part of a parallel loop bipolar electrode according to an embodiment of the invention.

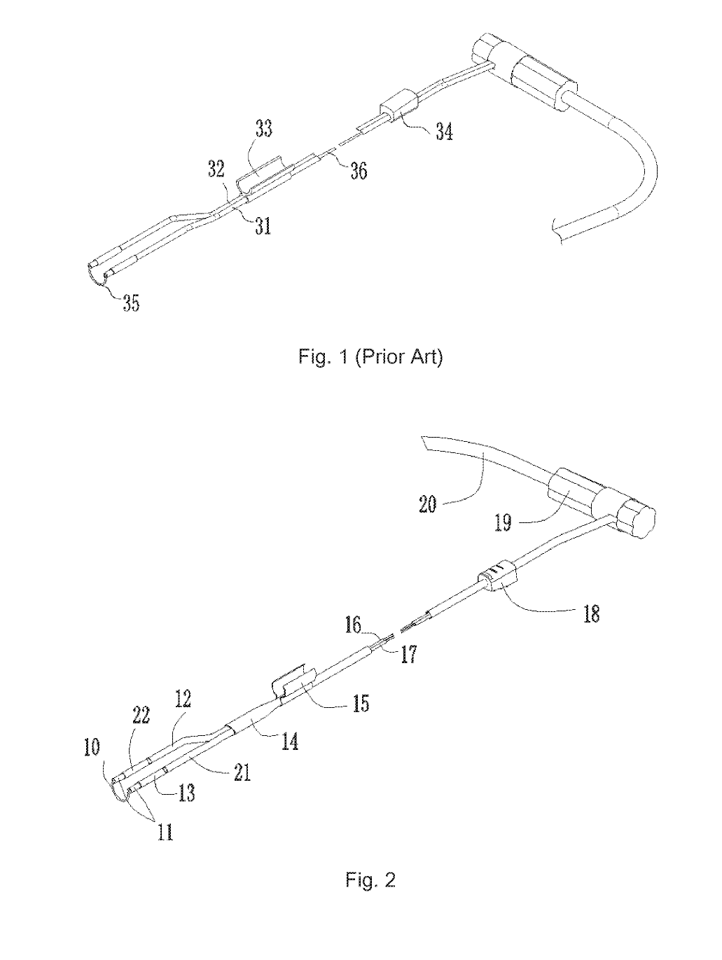

### DETAILED DESCRIPTION OF THE EMBODIMENTS

The embodiment provides a novel left-right parallel loop bipolar electrode, which, as shown in FIG. 2 to FIG. 5, includes:

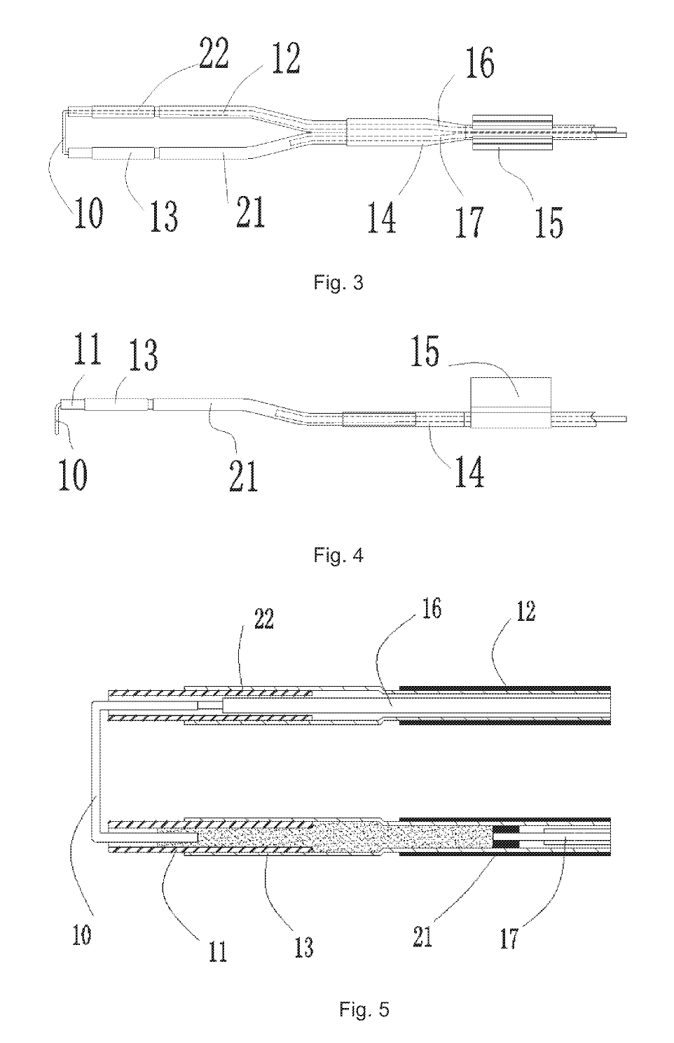

a positive electrode 10, two ceramic insulating tubes 11, a metal sleeve 13, a rigid tube 22, two insulating sleeves 12 and 21 which are forked by separation at one end and folding at the other end, a metal straight tube 14 with an enlarged opening, an electrode fixing clip 15, wires 16 and 17 with insulating layers, a waterproof plug 18, a binding post 19 and a double-core wire 20. The metal sleeve 13 and the rigid tube 22 are insulated from the metal straight tube 14 through a fork formed by the forked insulating sleeves 12 and 21 respectively (see details in FIG. 3 and FIG. 4).

The ceramic insulating tubes 11 are fixed at front ends of the metal sleeve 13 and the rigid tube 22 respectively, one end of the positive electrode 10 is connected with one wire 16 in the rigid tube 22 through one ceramic insulating tube 11, the other end is supported in the ceramic insulating tube 11 corresponding to the metal sleeve 13, the other wire 17 is welded with the corresponding metal sleeve 13, and tail peripheries of the metal sleeve and the rigid tube are sleeved with the insulating sleeves 12 and 21 so as to be insulated from the metal straight tube 14 respectively (see details in FIG. 4). The wires 16 and 17 penetrate through the metal straight tube 14. High-frequency energy passes through the positive electrode 10 in the rigid tube 22 and flows through the metal sleeve 13 to form a loop.

At a front section of the left-right parallel loop bipolar electrode of the embodiment, rear sections of the ceramic insulating tubes 11 are sleeved and covered by front sections of the metal sleeve 13 and the rigid tube 22, and front sections of the ceramic insulating tubes 11 are exposed; and rear sections of the metal sleeve 13 and the rigid tube 22 are sleeved and covered by the insulating sleeves 12 and 21, so that front sections of the metal sleeve 13 and the rigid tube 22 are exposed. Wherein the front section, which is exposed, of the metal sleeve 13 is exposed in a saline water environment when being used, its surface area is a surface area of a loop electrode, and a surface area of the positive electrode 10 is a surface area of a semicircular positive electrode exposed in the saline water environment. Preferably, the surface area of the loop electrode is 5 times to 16 times the surface area of the semicircular positive electrode.

The binding post 19 is arranged at a tail end of the metal straight tube 14, the double-core wire 20 extends from the binding post 19, and a plug is further plugged into a tail end of the binding post. The wires 16 and 17 are correspondingly connected with the double-core wire 20 after penetrating through the metal straight tube 14.

The electrode fixing clip 15 is welded on an outer wall of the metal straight tube, and is used for matching with a rod of an endoscope.

The waterproof plug 18 is closed attached to the outer wall of the metal straight tube 14 in a sleeving manner, and its specific position is between the electrode fixing clip 15 and the binding post 19. Specifically, the outer wall of the metal straight tube 14 is round, and a round hole is formed in the waterproof plug 18, so that the phenomenon of water leakage caused by an irregularly-shaped hole of a waterproof plug in the background may be avoided.

Preferably, the positive electrode is arranged to be a semicircular external spading shape, and it forms an angle of preferably 110-160 degrees with the metal sleeve.

Preferably, the positive electrode is arranged to be a semicircular internal hooking shape, and it forms an angle of preferably 70-90 degrees with the metal sleeve.

In fact, the technical solutions to be protected by the invention are not limited to the abovementioned embodiment, and there may also be multiple transformations based on the concept of the invention, for example:

the positive electrode 10 is usually semicircular, and may also be cylindrical, spade-shaped and the like.

The rigid tube 22 does not serve as the loop electrode, and may adopt an insulating material, and if the insulating material is adopted as the rigid tube 22, the insulating sleeve 12 may be or may not be adopted. For example: the rear section of the insulating rigid tube 22 is sleeved with no insulating sleeve, and instead, is folded with the rear section of the insulating sleeve, and a periphery of a folded part is sleeved and covered by the enlarged opening of the metal straight tube 14.

It is important to note that those skilled in the art may also make a plurality of transformations and improvements without departing from the concept of the invention, and these transformations and improvements shall all fall within the scope of protection of the invention. Therefore, the scope of protection of the invention should be subject to the appended claims.

1. A left-right parallel loop bipolar electrode, comprising:
a positive electrode, two insulating tubes, a metal sleeve serving as a negative electrode, a rigid tube, two insulating sleeves which are forked by separation at one end and folding at the other end, a metal straight tube with an enlarged opening, two wires with insulating layers, a double-core wire and a binding post, wherein
at a front-section part of the bipolar electrode, the metal sleeve and the rigid tube are arranged in parallel at an interval on left and right, the two insulating tubes are fixed at heads of the metal sleeve and the rigid tube respectively, one end of the positive electrode is connected with the wire, led from the rigid tube, with the insulating layer in the corresponding insulating tube, and the other wire with the insulating layer is welded with the metal sleeve; rear sections of the metal sleeve and the rigid tube are arranged in the two insulating sleeves in a sleeving manner respectively; at a rear-section part of the bipolar electrode, rear sections, which are folded, of the two insulating sleeves are sleeved and covered at the enlarged opening of the metal straight tube; a tail end of the metal straight tube is connected with the binding post, the binding post is connected with the double-core wire, and the two wires with the insulating layers penetrate through the metal straight tube to be correspondingly connected with the double-core wire respectively; the other end of the positive electrode is fixed in the other insulating tube; and
high-frequency energy forms a loop in a working medium through the two wires with the insulating layers in the metal straight tube.

2. (canceled)

3. The left-right parallel loop bipolar electrode according to claim 1, further comprising an electrode fixing clip, which is welded on an outer surface of the metal straight tube and used for matching with a rod of an endoscope.

4. The left-right parallel loop bipolar electrode according to claim 1, wherein an external diameter of the metal sleeve is 1 mm-1.7 mm, and an external diameter of the metal straight tube is 1.5-2.2 mm.

5. The left-right parallel loop bipolar electrode according to claim 4, wherein an exposed front-section part, uncovered by the insulating sleeve, of the metal sleeve is a loop electrode, and a surface area of the loop electrode is 5 to 16 times an electrode surface area of the cambered positive electrode.

6. The left-right parallel loop bipolar electrode according to claim 1, wherein a shape of the positive electrode is a semicircle, a cylinder or a spade.

7. The left-right parallel loop bipolar electrode according to claim 1, wherein the insulating tubes are ceramic insulating tubes.

8. A left-right parallel loop bipolar electrode, comprising:
a positive electrode, two insulating tubes, a metal sleeve serving as a negative electrode, an insulating rigid tube, an insulating sleeve, a metal straight tube with an enlarged opening, two wires with insulating layers, a double-core wire and a binding post;
at a front-section part of the bipolar electrode, the metal sleeve and the rigid tube are arranged in parallel at an interval on left and right, the two insulating tubes are fixed at heads of the metal sleeve and the insulating rigid tube respectively, one end of the positive electrode is connected with the wire, led from the rigid tube, with the insulating layer in the corresponding insulating tube, and the other wire with the insulating layer is welded with the metal sleeve; a rear section of the metal sleeve is arranged in the insulating sleeve in a sleeving manner; at a rear-section part of the bipolar electrode, a rear section of the insulating rigid tube is folded with a rear section of the insulating sleeve, and its periphery is sleeved and covered at the enlarged opening of the metal straight tube; a tail end of the metal straight tube is connected with the binding post, the binding post is connected with the double-core wire, and the two wires with the insulating layers penetrate through the metal straight tube to be correspondingly connected with the double-core wire respectively; the other end of the positive electrode is fixed in the other insulating tube; and
high-frequency energy forms a loop in a working medium through the two wires with the insulating layers in the metal straight tube.

9. The left-right parallel loop bipolar electrode according to claim 8, further comprising an electrode fixing clip, which is welded on an outer surface of the metal straight tube and used for matching with a rod of an endoscope.

10. The left-right parallel loop bipolar electrode according to claim 8, wherein an external diameter of the metal sleeve is 1 mm-1.7 mm, and an external diameter of the metal straight tube is 1.5-2.2 mm.

11. The left-right parallel loop bipolar electrode according to claim 10, wherein an exposed front-section part, uncovered by the insulating sleeve, of the metal sleeve is a loop electrode, and a surface area of the loop electrode is 5 to 16 times an electrode surface area of the cambered positive electrode.

12. The left-right parallel loop bipolar electrode according to claim 8, wherein a shape of the positive electrode is a semicircle, a cylinder or a spade.

13. The left-right parallel loop bipolar electrode according to claim 8, wherein the insulating tubes are ceramic insulating tubes.
